(19) United States
(12) Patent Application Publication
(10) Pub. No.: US 2010/0062989 A1
(43) Pub. Date: Mar. 11, 2010

(54) OPTIMAL BIOLOGICAL MARKER FOR THE BIOLOGICAL POTENCY OF Emblica Officinalis Gaertn. (AMLA) FRUIT-METHODS AND PRODUCTS THEREOF

(76) Inventors: Muhammed Majeed, Piscataway, NJ (US); Beena Bhat, Bangalore (IN); Atul N. Jadhav, Bangalore (IN); Jyotish S. Srivastava, Bangalore (IN); Kalyanam Nagabhushanam, Piscataway, NJ (US)

Correspondence Address: SABINSA CORPORATION, 70 ETHEL ROAD WEST, UNIT 6, PISCATAWAY, NJ, 08854, US

(21) Appl. No.: 12/353,381

(22) Filed: Jan. 14, 2009

Related U.S. Application Data
(63) Continuation-in-part of application No. 12/208,024, filed on Sep. 10, 2008.

(51) Int. Cl. A61K 31/7012; A61P 25/28; C07H 15/18

(52) U.S. Cl. 514/25; 536/18.2

(57) ABSTRACT

Disclosed is the most optimal biomarker for the BIOLOGICAL POTENCY of Emblica Officinalis Gaertn. (Amla) fruit and products standardized for 5% and above w/w of the said biomarker. Further, the products described herein contain from about 0.00010% to about 4% of free ascorbic acid depending on the raw material used. The optimal biomarker for amla described herein above is represented by STR#1.

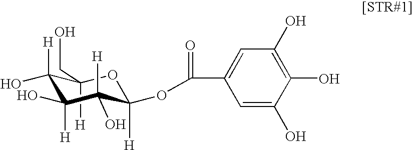

### CROSS-REFERENCE TO RELATED APPLICATIONS

This application is a continuation-in-part of application Ser. No. 12/208,024 filed on Sep. 10, 2008 at the United States Patents and Trademarks Office, the content of which is incorporated herein by reference.

### BACKGROUND OF THE INVENTION

1. Field of the Invention

The invention in general relates to Emblica Officinalis Gaertn. (Amla) fruit. More specifically, the present invention relates to novel optimal biomarkers for the biological potency of amla fruits and products thereof.

2. Description of Prior Art

The fruits of E. officinalis are reported to contain hydrolysable tannins, emblicanin A and emblicanin B, along with pedunculagin and punigluconin [Ghosal, S.; Tripathi, V. K.; Chauhan, S. Active constituent of Emblica officinalis: part I—the chemistry and antioxidant effects of two new hydrolysable tannins, emblicanin A and B. Indian J. Chem. 1996, 35B, 941-948]. Important patent references in this regard include:
A. Process for obtaining a stabilized antioxidant formulation from Emblica officinalis fruit comprising extracting the finely pulped fruit with a dilute aqueous or alcoholic water salt solution at a temperature of about 70° C. 5° C. to form an extract containing solution, filtering, and drying to provide the desired antioxidant blend as a powder has been discussed in WO/2000/048551, U.S. Pat. No. 6,124,268, U.S. Pat. No. 6,235,721 to Ghosal, Shibnath.
B. The main constituents from the pericarp of Emblica officinalis discussed here include
   1. Emblicanin-A: 2,3-di-O-galloyl-4,6-(S)-hexahydroxydiphenoyl-2-keto-glucono-lactone;
   2. Emblicanin-B: 2,3,4,6-bis-(S)-hexahydroxydiphenoyl-2-keto-glucono-lactone;
   3. Punigluconin: 2,3-di-O-galloyl4,6-(S)-hexahydroxydiphenoyl gluconic acid;
   4. Pedunculagin: 2,3,4,6-bis-(S)-hexahydroxydiphenoyl-D-glucose; 5. Rutin: 3′,4′,5,7-tetrahydroxyflavono-1,3-O-rhamnoglucoside;
   5. Gallo-ellagitannoids;
   6. Gallic acid and
   7. Ellagic acid.
C. Sun protective and skin lightening dosage forms of Emblica officinalis extract comprising Emblicanin A, Emblicanin B, Pendunculagin and Punigluconin, preferably in an amount of >40% by weight of the extract have been discussed in US20050089590, EP1560561, US20030198612 and US20040086560.

Amla fruits were to be considered rich sources of ascorbic acid until Ghosal et al. questioned its presence in 1996 [Ghosal, S.; Tripathi, V. K.; Chauhan, S. Active constituent of Emblica officinalis: part I—the chemistry and antioxidant effects of two new hydrolysable tannins, emblicanin A and B. Indian J. Chem. 1996, 35B, 941-948].

Similar views on ascorbic acid contents in Emblica officinalis to bring about the biological effects of amla were discussed in “A much higher concentration of Ascorbic acid is required to effect the same inhibition as Amla”—S. M. Khopde et al., Current Science, Vol. 81, No. 2, 25 Jul. 2001.

However, in 2006, Scartezzini et al. proposed a reliable HPLC-DAD for the identification and quantification of ascorbic acid and further indicated that high antioxidant activity is due to a large percentage of the presence of ascorbic acid (Scartezzini, P.; Antognoni, F.; Raggi, M. A.; Poli, F.; Sabbioni, C. Vitamin C content and antioxidant activity of the fruit and of the Ayurvedic preparation of Emblica officinalis Gaertn. J. Ethnopharm. 2006, 104, 113-118).

Recently, Raghu et al. compared ascorbic acid content of the fruits by conventional calorimetric estimation and specific enzymatic method and as the o-phenylene diamine derivative of dehydroascorbic acid and found contents of 34-38 mg of vitamin C equivalent to 100 g of fresh weight (Raghu, V.; Platel, K.; Srinivasan, K. Comparison of ascorbic acid content of Emblica officinalis fruits determined by different analytical methods. J. Food Compos. Anal. 2007, 20, 529-533.)

Zhang et al have reported the principal phenolic constituents of Emblica officinalis fruit juice as mucic acid 2-O-gallate, mucic acid 1,4-lactone 2-O-gallate and mucic acid 1,4-lactone 5-O-gallates. These along with 1-O-galloyl-b-D-glucose (β-glucogallin) have been suggested as possible antioxidants in the juice together with vitamin C.

The inventors of the present invention revisited the amla chemistry to identify and confirm the essential biomarkers of the fruit that confer its vital properties.

Accordingly, it is the principle object of the present invention to investigate thoroughly the chemistry of amla and unravel unknown data if any and also confirm the essential biomarkers of the fruit that confer its vital properties.

It is also another object of the present invention to develop a product standardized in terms of the most optimal biomarker molecule as identified by the chemical studies on amla fruit.

The present invention fulfills the aforesaid objectives and provides further related advantages.

### SUMMARY OF THE INVENTION

The present invention relates to the most optimal biomarker for the biological potency of Emblica Officinalis Gaertn. (Amla) fruit and products standardized for 5% and above w/w of the said biomarker represented by STR#1. Further, the products described herein above contain from about 0.00010% to about 4% of free ascorbic acid depending on the raw material used. In specific embodiment of the present invention, the product standardized for 5% and above w/w of the said biomarker represented by STR#1 represents an aqueous extract from Emblica Officinalis Gaertn. fruit (Amla fruit). More specifically, the aqueous extract also comprises from about 50% to greater than 50% total gallates including mucic acid 1,4-lactone 5-O-gallate, mucic acid 2-O-gallate, mucic acid 6-Methyl ester 2-O-gallate, mucic acid 1-Methyl ester 2-O-gallate and ellagic acid.

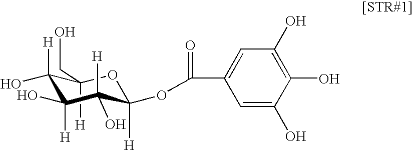

The advantages of the present invention include
1. The invention that β-glucogallin is the most optimal biomarker for the efficiency of Emblica Officinalis Gaertn. (Amla) fruit along with co-eluting mucic acid gallates, rather than previously reported hydrolysable tannins such as Emblicanin A and Emblicanin B, which have been shown absent in amla fruit by the current inventors.
2. The demonstration of poor, variable presence of ascorbic acid adding new dimensions to the myth that amla is rich in ascorbic acid and ascorbic acid is the most optimal biomarker for amla efficacy.
3. A product comprising the compound represented by STR#1, said compound used as the most optimal biomarker for the efficiency of Emblica Officinalis Gaertn. (Amla) fruit, wherein said product is standardized to contain 5% and above w/w of the biomarker represented by STR#1 and from about 0.00010% to about 4% free ascorbic acid depending on the raw material used.

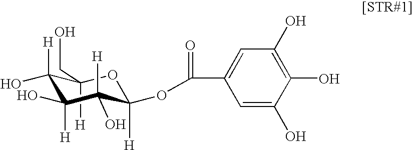

Other features and advantages of the present invention will become apparent from the following more detailed description, taken in conjunction with the accompanying drawings, which illustrate, by way of example, the principle of the invention.

### BRIEF DESCRIPTION OF THE DRAWINGS

FIG. 1 shows the preparative HPLC chromatogram (at 280 nm) of freeze-dried aqueous extract of E. officinalis fruits.

FIG. 2 shows the TLC of isolated peaks with ascorbic acid (spots from left to right: 1, fraction 1; 2, fraction 2; AA, ascorbic acid; 3, fraction 3; 4, fraction 4, 5, FRACTION 5, 6, FRACTION 6, 7, FRACTION 7)

FIG. 3 shows the HPLC chromatogram of (A) ascorbic acid (RT 4.57 min) and (B) PEAK 3 (STR#1) (RT 12.6 min).

FIG. 4 shows the HPTLC profile of E. officinalis fruit extract.

FIG. 5 shows the compounds from E. officinalis fruit extract: 1. β-glucogallin; 2. mucic acid 1,4-lactone 5-O-gallate; and 3. mucic acid 2-O-gallate.

FIG. 6 shows the HPLC chromatogram of (A) Ascorbic Acid With Retention Time 4.57 Min; (B) Fraction 1; (C) Peak 1 Spiked With Ascorbic Acid.

FIG. 7 shows the LC-ESIMS of Peak 1.

FIG. 8 shows the comparative anti-oxidant potential in terms of ORAC values, where greater the ORAC value greater is the anti-oxidant activity.

FIG. 9 shows the comparative anti-oxidant potential in terms of DPPH scavenging activity, where lower the IC50 values, greater is the Antioxidant activity.

FIG. 10 shows the comparative UVB protection values [EC₅₀ μg/ml] (represented on Y axis) of A=Ascorbic acid; B=β-glucogallin and C=Mucic acid gallates (represented on X Axis)

FIG. 11 shows the HPLC chromatogram of β-glucogallin and ascorbic acid in amla juice.

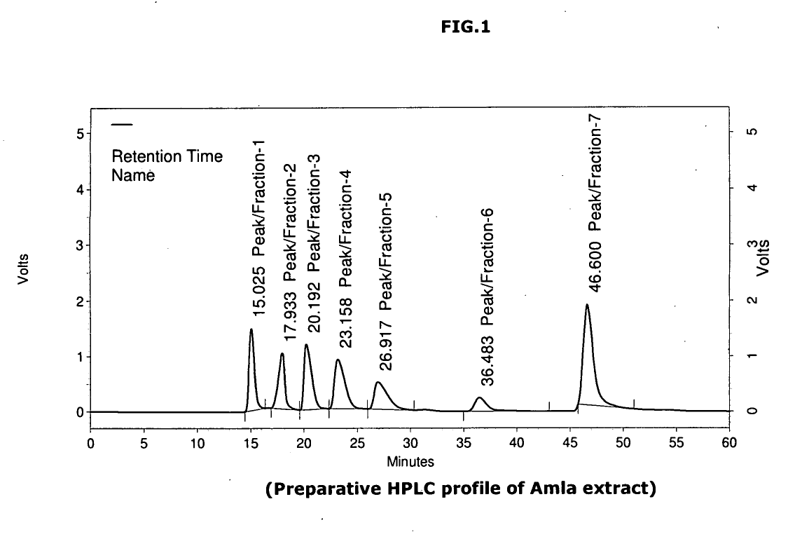

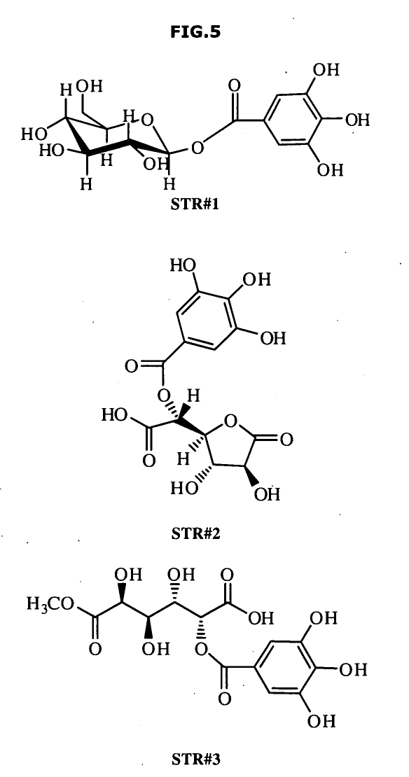

DESCRIPTION OF THE MOST PREFERRED EMBODIMENT (FIGS. 1 to 7)

In the most preferred embodiment, the present invention relates to the most optimal biomarker for the biological potency of Emblica Officinalis Gaertn. fruit (Amla fruit) and products standardized for 5% and above w/w of the said biomarker represented by STR#1. Further, the products described herein above contain from about 0.00010% to about 4% of free ascorbic acid depending on the raw material used.

In specific embodiment of the present invention, the product standardized for 5% and above w/w of the said biomarker represented by STR#1 represents an aqueous extract from Emblica Officinalis Gaertn. fruit (AmIa fruit). More specifically, the aqueous extract also comprises from about 50% to greater than 50% total gallates including mucic acid 1,4-lactone 5-O-gallate represented by STR#2, mucic acid 2-O-gallate represented by STR#3, mucic acid 6-Methyl ester 2-O-gallate represented by STR#4, mucic acid 1-Methyl ester 2-O-gallate represented by STR#5 and ellagic acid represented by STR#6.

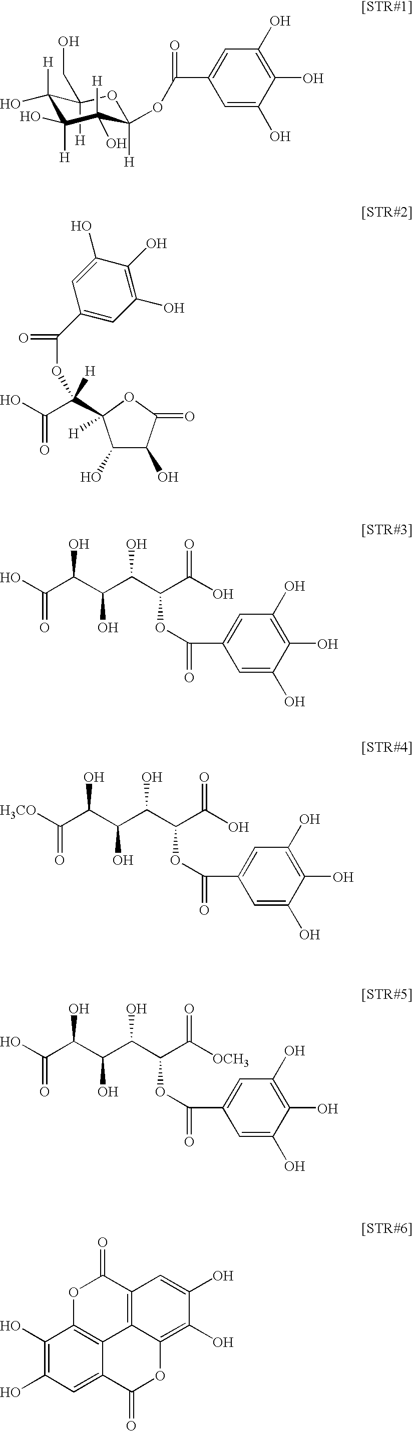

The aforesaid most preferred embodiments are explained in detail by specific examples described herein below.

### EXAMPLE I

### CHEMICAL ANALYSIS OF Emblica Officinalis FRUITS

The present inventors separated the extract of Emblica officinalis into seven major fractions using Preparative Shimadzu HPLC system equipped with Class Vp software with binary gradient (LC8A) pump, UV-VIS dual wavelength detector (SPD-10AVP), system controller (SCL-10AVp) and Rheodyne injector 7725i with 5 mL sample loop. Separation was done on Phenomenex Jupiter C18 column 300A(250×50 mm, 15 μm) with mobile phase A—water (0.1% formic acid) and B—methanol. A gradient was used as 0-35 min 5% B, 35-40 min 100% B, 40-50 min. 100% B, 50-51 min. 5% B and 51-60 min. 5% B. Flow rate was 35 ml/min and monitoring at 240 and 280 nm.

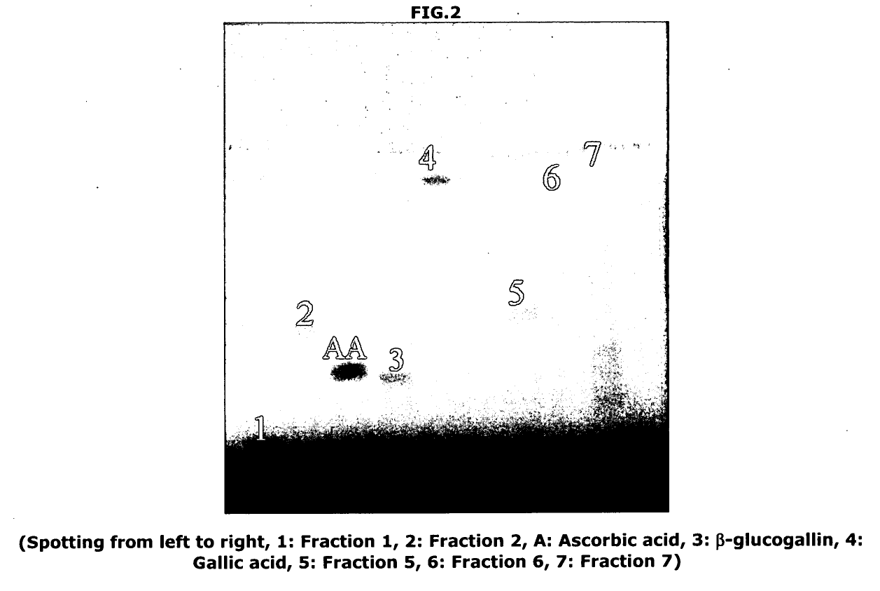

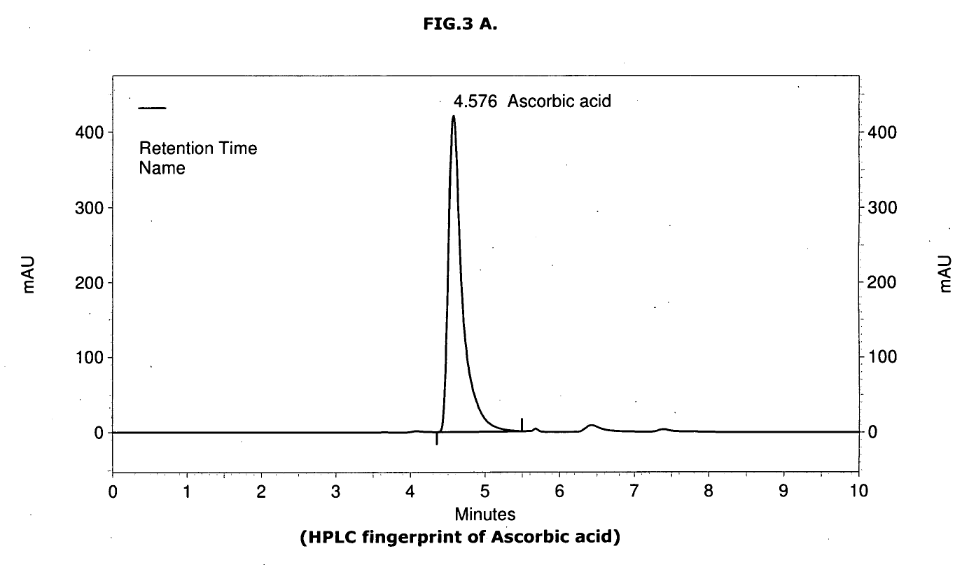

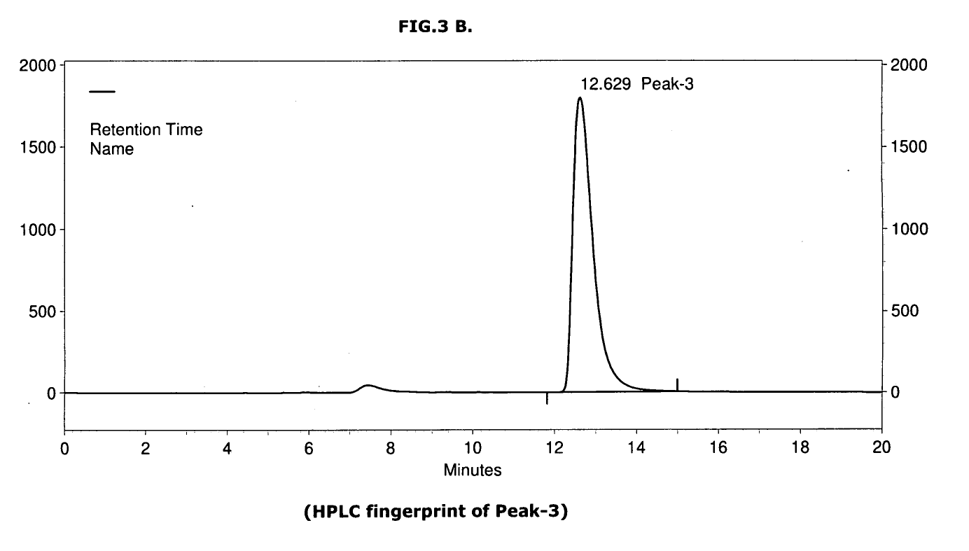

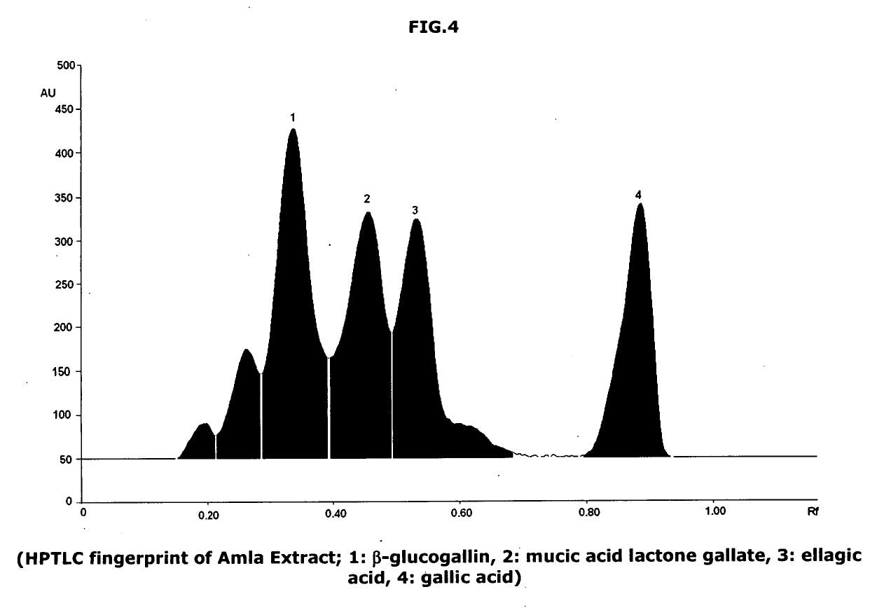

FIG. 1 shows the preparative HPLC profile of Emblica officinalis as discussed herein above. FIG. 2 shows HPTLC profile of the seven peaks along with standard ascorbic acid. FIG. 3 shows the different retention times for ascorbic acid and peak 3. The HPTLC profile as seen in FIG. 4 elucidated by the present inventors perfectly matches with reported HPTLC profile for Emblica officinalis [J. Sep. Sc. 2000, 30, 1250-1254]. Peak 3 obtained by the present inventors indeed corresponded to Emblicanin A by TLC profiling (FIG. 2). Further HPLC profiling (FIG. 3) of Peak 3 reported by the present inventors and ascorbic acid, clarify that peak 3 is not ascorbic acid owing to the variant retention time values of ascorbic acid being 4.6 and Peak 3 being 1 2.6. Identification of peak 3 isolated by the current inventors using ¹H, ¹³C NMR & Mass yielded a structure (STR#1) different from the one reported for Emblicanin A in prior art (STR#001). STR#1 corresponded to 1-O-galloyl-β-D-glucose(β-glucogallin).

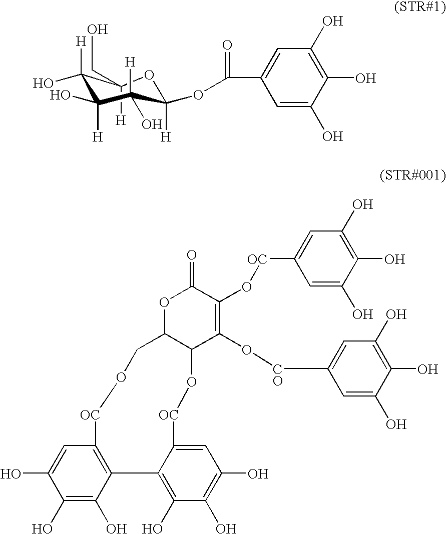

Under stringent conditions of temperature and light, the present inventors isolated, freeze dried and subjected Peak 2 of FIG. 1 to NMR studies. ¹H, ¹³C NMR & Mass yielded a structure (STR#2) for Peak 2. However peak 2 corroborated with emblicanin B in HPTLC studies. This led to conclusion that Emblicanin B, represented by STR#002 is actually Peak 2 with correct structure (STR#2) indicating it to be Mucic acid 1,4-lactone 5-O-gallate.

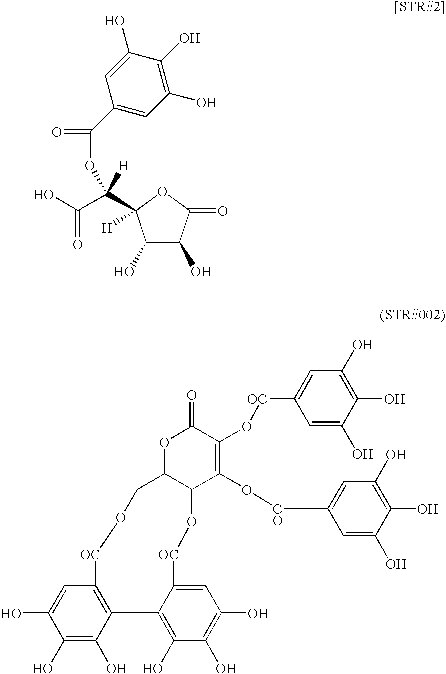

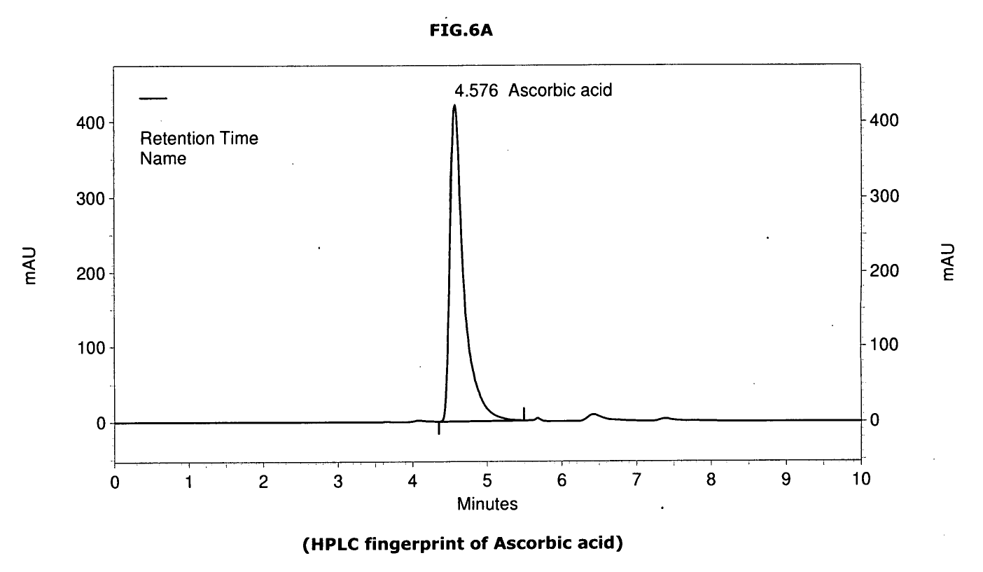

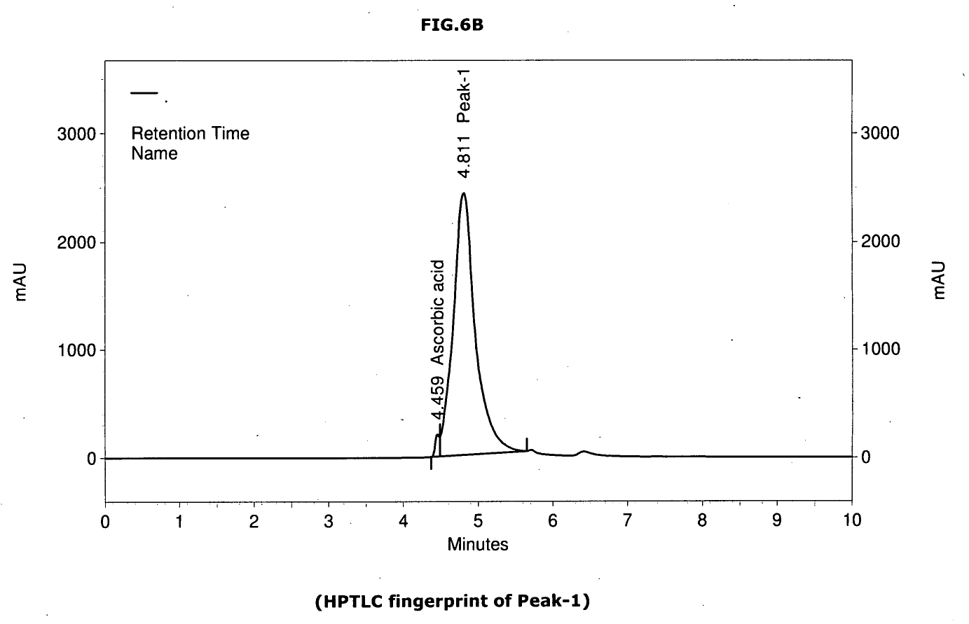

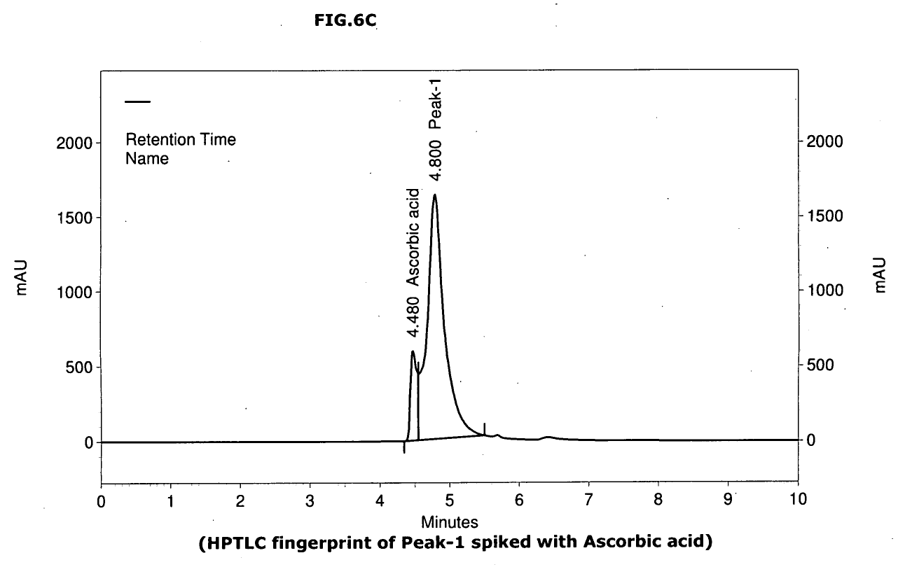

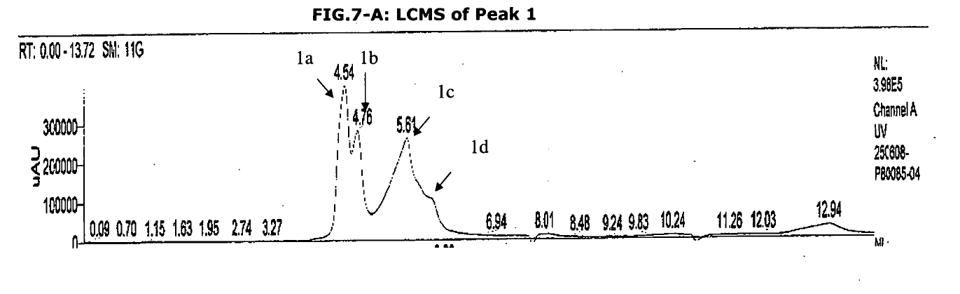

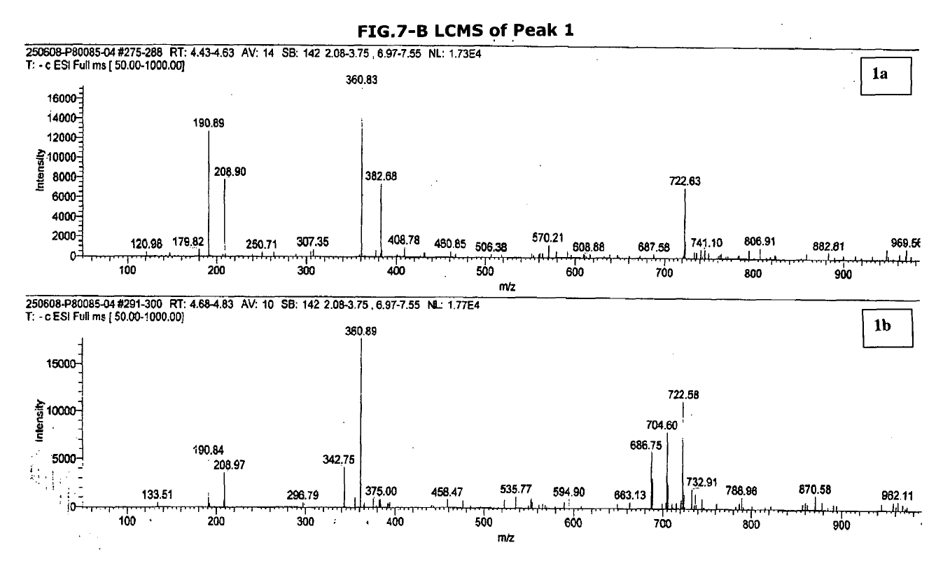

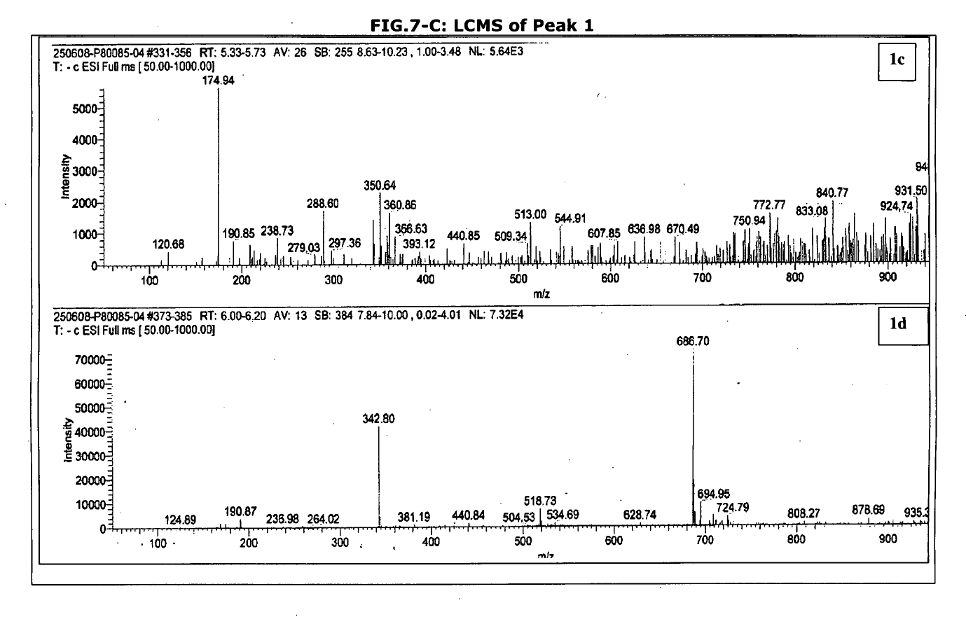

Ascorbic acid has been estimated previously by various methods. Retention time of Ascorbic acid (FIG. 6A) and peak 1 (FIG. 6B) were nearly same. Standard ascorbic acid when spiked with peak 1 (FIG. 6C) was found to elute nearly at the same retention time. To evaluate the presence of ascorbic acid, peak 1 isolated by preparative HPLC from Emblica officinalis extract was freeze dried, and was successfully separated into mixture of four constituents on reverse phase HPLC and LCMS analysis was done using the same method. However on LCMS studies ascorbic acid presence was revealed FIG. 7. The ¹³C NMR of peak 1 established it to contain (STR#3), mucic acid 2-O-gallate.

### EXAMPLE 2

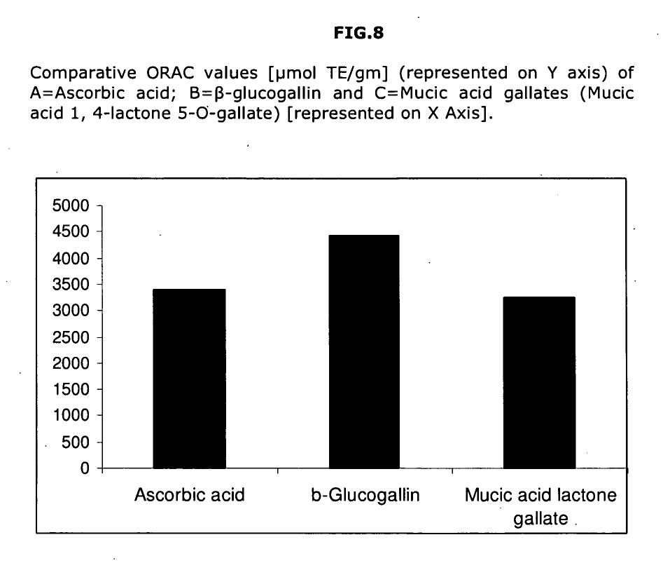

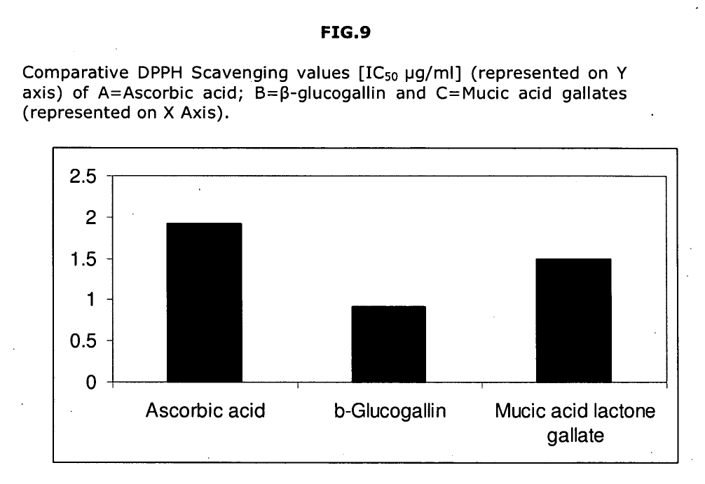

### BIOLOGICAL POTENCY OF Emblica Officinalis FRUITS IN TERMS OF ANTI-OXIDANT ACTIVITY (FIGS. 8 and 9)

I. Oxygen Radical Absorbance Capacity (ORAC)—ORAC value is a measure of inhibition of peroxyl radicals. ORAC value is represented as Trolox equivalents where the product is compared with Trolox, a standard inhibitor of peroxyl radicals. Higher the ORAC value, higher is the peroxyl radical inhibition. ORAC value is represented as Trolox equivalents per gm or liter by calculating the area under the curve formed by the inhibition of quenching of fluroscein dye induced by the peroxyl radical generator, azobis.

II. DPPH scavenging—Generation of free radicals in skin due to various stress conditions such as UV exposure, pollution, ageing etc, result in induction of melanin synthesis. The quenching of the stable free radical Diphenylpicryl hydrazyl (DPPH) in a methanolic reaction mixture by the antioxidant is an indication of the antioxidant potential.

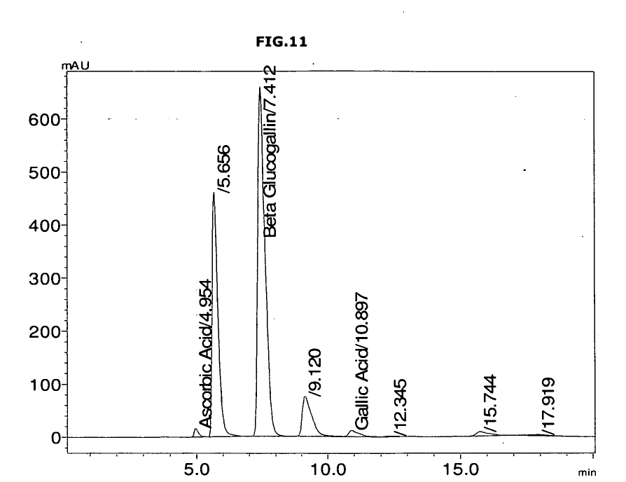

In terms of both DPPH scavenging assay and ORAC values (FIGS. 8 and 9), it is evident that β-glucogallin is a much better anti-oxidant than ascorbic acid or mucic acid gallates. With proof of (i) the absence of hydrolysable tannins such as Emblicanin A and Emblicanin B (ii) higher relative content of β-glucogallin over ascorbic acid in amla juice (FIG. 11, Table A); (iii) from about 0.00010% to a maximum of about 4% of free ascorbic acid in amla; (iv) the enhanced anti-oxidant potential of β-glucogallin over ascorbic acid and mucic acid gallate (Mucic acid 1,4-lactone 5-O-gallate); and (v) the relative unstable nature of mucic acid gallates, it is clear that the biological activity of amla is concentrated in β-glucogallin which represents the most optimal biological marker.

TABLE A

| | Collection Amla | β-Glucogallin | Ascorbic acid |
|---|---|---|---|
| S. No. | Samples | mg/ml | mg/ml |
| 01 | Uttar Pradesh | 6.20 | 2.19 |
| | (Krishna + | | |
| | Narendra) | | |
| 02 | Maharastra | 6.52 | 2.85 |
| | (Krishna) | | |
| 03 | Tamilnadu | 10.62 | 3.04 |
| | (BSR-1) | | |
| 04 | Rajasthan (Chakia) | 9.52 | 2.85 |
| 05 | Karnataka | 6.35 | 2.74 |
| | (Kanchan) | | |

### EXAMPLE III

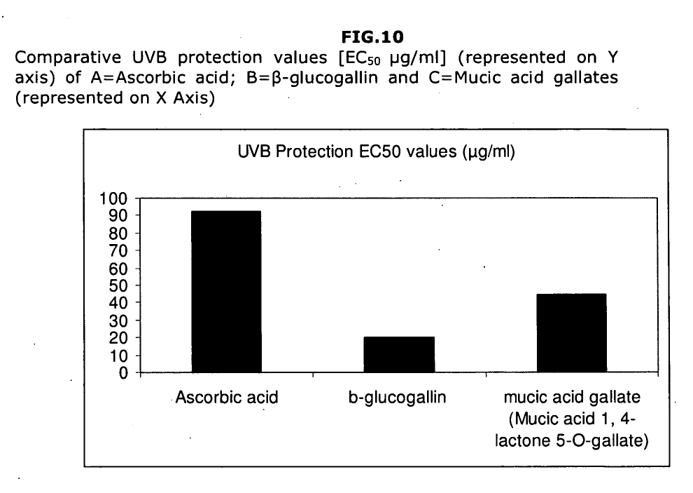

### BIOLOGICAL POTENCY OF Emblica Officinalis FRUITS IN TERMS OF UVB PROTECTANT ACTIVITY (FIG. 10)

Varying concentrations of the test sample in the cell culture medium were added to the 24 hr monolayers of cells in a 96 well flat bottomed clear plates and then exposed to UV irradiation dose of 0.05 J cm-2. Each concentration was applied with ‘n’ value of 12. The plate layout was made in such a way that six of the replicates were exposed to UV and six unexposed. The unexposed region of the plate was covered by aluminum foil. After exposure, the cells were incubated in a CO2 incubator for 48 hrs and developed by the NRU staining techniques to analyze the cell viability. The percentage of UV protection was calculated with respect to the cytotoxicity in treated cells as compared to that of the untreated cells.

UV source: UV lamp of 14.7 W, 0.3 A, 55V with an intensity of 33.3 μW cm-2 was obtained from Sankyo Denki Co., Ltd, Japan.

### EXAMPLE III

### PRODUCT (SABERRY™) REPRESENTED BY THE PRESENT INVENTION (Table B)

TABLE B

| PARAMETER | | RESULTS OF ANALYSIS |
|---|---|---|
| DESCRIPTION | | Beige colored odorless |
| | | hygroscopic powder |
| IDENTIFICATION | | To complies by TLC |
| SOLUBILITY | | Not less than 90.0% w/w |
| Water solubles (% w/w) | | |
| (1% w/v solution in water) | | |
| Residue on Ignition | | Not more than 15% w/w |
| Moisture content in KF (% w/w) | | Not more than 5% w/w |
| Total Heavy Metals | | Not more than 20 ppm |
| Lead | | Not more than 3 ppm |
| Arsenic | | Not more than 1 ppm |
| Cadmium | | Not more than 1 ppm |
| Mercury | | Not more than 0.1 ppm |
| Tapped Bulk Density | | 0.45 g/ml to 0.80 g/ml |
| Loose bulk density | | 0.35 g/ml to 0.60 g/ml |
| Assay on | Content of β-glucogallin | Greater than 5% w/w |
| anhydrous basis | by HPLC | |
| | Content of gallic acid by | Not more than 2% w/w |
| | HPLC | |
| | Total gallates by UV | Greater than 50% w/w |

Table C-SABERRY™: Antioxidant Profile

TABLE C

| | ORAC | | ORAC | HORAC | | | SOAC |
|---|---|---|---|---|---|---|---|
| | hydro | ORAC lipo | total | (μm | NORAC | SOD | (μm |
| Product | (μm TE/g) | (μm TE/g) | (μm TE/g) | CAE/g) | (μm TE/g) | kunitSODeq/g | VITE/g) |
| SABERRY ™ | 2678 | 4 | 2682 | 345 | 904 | 102 | 1351 |

1. ORAC ANALYSIS—Oxygen Radical Absorbance Capacity
2. ORAC hydro—Water soluble antioxidant capacity.
3. ORAC lipo—Lipid soluble antioxidant capacity.
4. NORAC—Peroxynitrite absorbance capacity
5. HORAC—Hydroxyl radical absorbance capacity
6. SOD—Superoxide dismutase activity
7. SOAC—Singlet oxygen absorbance capacity

In-House Clinical Data on the Antioxidant Capacity of SABERRY™.

Study Design: Two healthy subjects and four unhealthy subjects (age: 40 to 50) were recruited to participate in this study. All healthy participants were in good health as determined by the following eligibility criteria: 1) no history of cardiovascular, hepatic, gastrointestinal, or renal disease; 2) no alcoholism; 3) no antibiotic or supplemental vitamin and/or mineral use for at least 4 wk before the start of the study and 4) no smoking. The unhealthy individuals were obese and frequent smokers.

A 10 mL blood sample (zero baseline samples) was obtained from fasting subjects at the start of the study, following which they were given 200 mg per day of SABERRY™ for a period of one month. Blood samples (10 mL) were collected again after one month. ORAC assay and ROS scavenging assay were conducted for all of the serum samples.

Oxygen Radical Absorbance Capacity (ORAC)

Principle: The Oxygen Radical Absorbance Capacity (ORAC) assay depends on the free radical damage to a fluorescent probe through the change in its fluorescence intensity. The change of fluorescence intensity is an index of the degree of free radical damage. In the presence of antioxidant, the inhibition of free radical damage, which is reflected in the protection against the change of probe fluorescence, is a measure of its antioxidant capacity against the free radical.

Procedure:

25 μl of each of the serum samples was pipetted into each well followed by 150 μl of 10×10⁻²M (final conc) AAPH reagent (2,2′-Azobis(2-amidinopropane)dihydrochloride) made in 75 mM potassium phosphate buffer (pH 7.4). Then 150 μl of disodium fluorescein dye (final conc.) 4.8×10⁻⁷M was added and mixed before the initial reading (f₀) was taken. Fluorescence reading were taken (Fluostar Optima Microplate Reader) at 485/520 nm after every 1 minute for 35 minutes (f₁ . . . f₃₅). 25 μl of phosphate buffer (75 mM) was pipetted instead of antioxidant in the blank. Trolox standard from 12.5-200 μM was also kept. The number of wells being used in the experiment should not exceed 20 to reduce the error due to time lag. Difference between duplicates also occurs due to the time lag and to avoid this, the experiment is to be repeated thrice with % CV not more than 15.

Calculation: The final ORAC values were calculated by using a quadratic regression equation (Y=a+bX+cX²) between the trolox concentration (Y) (μM) and the net area under the Fluorescence decay curve (X) and were expressed as micromoles of trolox equivalents per liter or per gram of sample (μmol TE/g or μmol TE/L).

The Area under curve AUC=(1+f₁/f₀+f₂/f₀+ . . . +f₃₅/f₀.) eq 1

Where f₀ is the initial fluorescence reading at 0 min and f₁ is the fluorescence reading after 1 min.

The data were analyzed by applying eq 1. The net AUC was obtained by subtracting the AUC of the blank from that of the sample. The value calculated using the net AUC of the sample and the quadratic regression equation was divided by the weight of the sample in g/L. The final value obtained is the ORAC value of the sample expressed as μmol trolox equivalents (TE)/g.

Result (Table D):

TABLE D

| | |
|---|---|
| | ORAC value |
| Health | (μmol trolox equivalents (TE)/g) |

| Subjects | condition | Before treatment | After treatment |
|---|---|---|---|
| Subject 1 | Healthy | 3100 ± 100 | 3210 ± 100 |
| Subject 2 | Healthy | 3520 ± 110 | 3690 ± 108 |
| Subject 3 | Unhealthy | 1312 ± 140 | 1600 ± 120 |
| Subject 4 | Unhealthy | 1223 ± 120 | 1552 ± 102 |
| Subject 5 | Unhealthy | 1556 ± 121 | 1842 ± 112 |
| Subject 6 | Unhealthy | 1426 ± 142 | 1782 ± 122 |

Note:
Higher ORAC value indicates better efficacy

Conclusion:

The serum ORAC values for healthy individuals did not vary significantly before and after treatment with SABERRY™. However, for unhealthy individuals, the serum ORAC values increased significantly in the range of 10-25%. In conclusion, SABERRY™ is good for the maintenance of the body's antioxidant potential. The reduced levels of antioxidant potential in unhealthy conditions were raised significantly and were not increased significantly in healthy conditions.

Reactive Oxyben Species (ROS) Scavenging Assay in Swiss 3T3 Fibroblast Cell Line

Principle: A cell permeable, non-fluorescent dye, DCFH-DA (dichloro fluorescein diacetate) after intracellularly getting de-esterified is converted to dichlorofluorescein upon oxidation by the reactive oxygen species. The scavenging activity of sample is indicated by the decrease in fluorescence (wavelength 485/520 nm).

Procedure: Serum samples (0.05 ml) in 96 well black flat-bottomed tissue culture plates were incubated with 100 μl of stock solution of DCFH-DA (2 mg/ml in ethanol, diluted to 100 times in phosphate buffered saline) for 1 hr at 37° C. and the fluorescence reading was taken at wavelength 485/520 nm (Fluostar optima microplate reader).

Calculation: The optical density (OD) measured is directly proportional to the ROS generated.

Result (Table E):

TABLE E

| | |
|---|---|
| Health | OD due to ROS generation |

| Subjects | condition | Before treatment | After treatment |
|---|---|---|---|
| Subject 1 | Healthy | 0.41 | 0.42 |
| Subject 2 | Healthy | 0.39 | 0.40 |
| Subject 3 | Unhealthy | 0.81 | 0.62 |
| Subject 4 | Unhealthy | 0.72 | 0.57 |
| Subject 5 | Unhealthy | 0.96 | 0.72 |
| Subject 6 | Unhealthy | 0.84 | 0.61 |

Conclusion:

The serum ROS levels for healthy individuals did not vary significantly before and after treatment with SABERRY™. However, for unhealthy individuals, the serum ROS levels decreased significantly in the range of 20-25%. In conclusion, SABERRY™ is good for reducing the excess ROS levels. The increased levels of ROS in unhealthy conditions were decreased significantly and were not affected in healthy conditions.

As an additional embodiment, the present invention also relates to the use of most optimal biomarker represented by STR#1 of Emblica Officinalis Gaertn. fruit (Amla fruit) and products standardized for 5% and above w/w of the said biomarker as peroxynitrite scavengers. Further, the products described herein above contain from about 0.00010% to about 4% of free ascorbic acid depending on the raw material used. In specific embodiments of the present invention, the product standardized for 5% and above w/w of the said biomarker represented by STR#1 represents an aqueous extract from Emblica Officinalis Gaertn. fruit (Amla fruit). More specifically, the aqueous extract also comprises from about 50% to greater than 50% total gallates including mucic acid 1,4-lactone 5-O-gallate represented by STR#2, mucic acid 2-O-gallate represented by STR#3, mucic acid 6-Methyl ester 2-O-gallate represented by STR#4, mucic acid 1-Methyl ester 2-O-gallate represented by STR#5 and ellagic acid represented by STR#6. In further specific embodiments, the present invention relates use of the aforesaid product for managing peroxynitrite-mediated damage occurring in Alzheimer's disease.

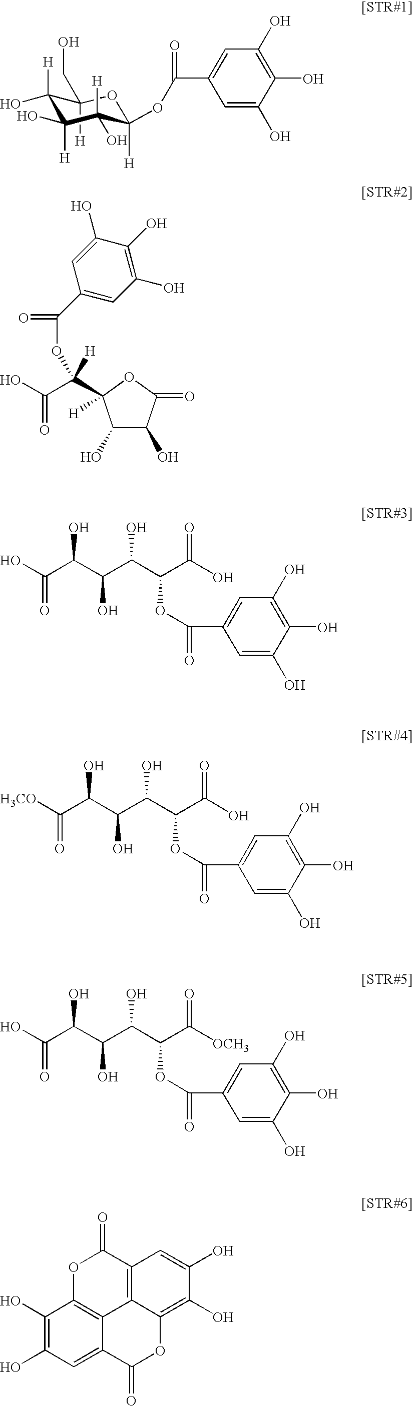

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A most optimal biomarker for the biological potency of Emblica Officinalis Gaertn. fruit (Amla fruit), said marker represented by STR#1.

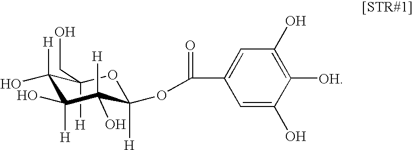

2. A product standardized for the compound represented by STR#1, said product comprising 5% and above w/w of the biomarker represented by STR#1.

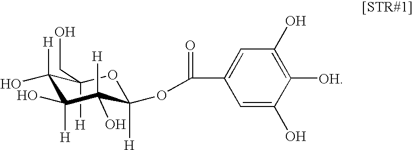

3. The product according to claim 2, wherein the said product is an aqueous extract of Emblica Officinalis Gaertn. fruit (Amla fruit).

4. The product according to claim 2, wherein the said product further comprises from about 0.00010% to about 4% of free ascorbic acid depending on the raw material used.

5. The product according to claim 3, wherein said product comprises from 50% to greater than 50% total gallates also including mucic acid 1,4-lactone 5-O-gallate represented by STR#2, mucic acid 2-O-gallate represented by STR#3, mucic acid 6-Methyl ester 2-O-gallate represented by STR#4, mucic acid 1-Methyl ester 2-O-gallate represented by STR#5 and ellagic acid represented by STR#6.

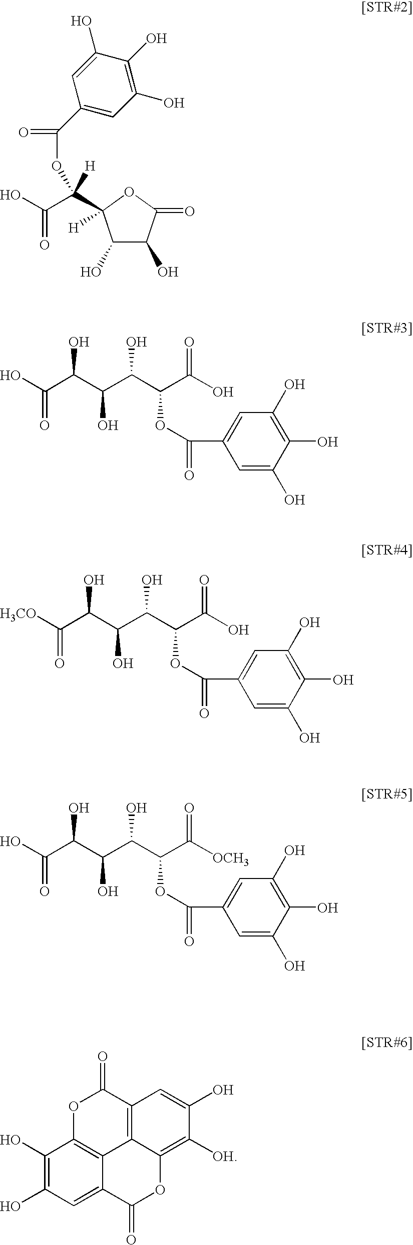

6. The product according to claim 2, wherein said product is useful for scavenging peroxynitrite radicals.

7. The product according to claim 2, wherein said product is useful for managing peroxynitrite-mediated damage occurring in Alzheimer's disease.
